(19) United States
(12) Patent Application Publication
(10) Pub. No.: US 2020/0113578 A1
(43) Pub. Date: Apr. 16, 2020

(54) PRESSURE CONTROLLERS FOR BLOOD FLOW RESTRICTION TRAINING AND OTHER APPLICATIONS

(71) Applicant: Ryan Osborn, Honolulu, HI (US)

(72) Inventor: Ryan Osborn, Honolulu, HI (US)

(21) Appl. No.: 16/598,990

(22) Filed: Oct. 10, 2019

Related U.S. Application Data
(60) Provisional application No. 62/744,854, filed on Oct. 12, 2018.

(51) Int. Cl. A61B 17/135

(52) U.S. Cl. CPC A61B 17/135; A61B 2017/00022

(57) ABSTRACT

Flexible air bladders and associated air assemblies provide compression and blood flow moderation in a variety of applications including blood flow restriction training (BFR) and limb compression for injury prevention. An integrated air assembly comprises a flexible manual pump, a check valve air intake, one-way valve and a pressure release valve. The user can properly position and fasten the air bladder around a limb using a special type of band, sleeve, or by wearing a piece of clothing containing strategically placed air bladders and integrated air assemblies. Each air assembly allows the user to easily and manually add air to the bladder by pressing on a designated area of the pump which causes the bladder to extend on the inward facing portion of the band, compression sleeve, or piece of clothing toward the limb until a desired pressure is achieved around the limb to support blood flow moderation.

### REFERENCE TO RELATED APPLICATIONS

This application claims priority to, and the benefit of, U.S. Provisional Patent Application Ser. No. 62/744,854, filed Oct. 12, 2018, the entire content of which is incorporated herein by reference.

### FIELD OF THE INVENTION

This invention is directed to flexible air bladders and associated air assemblies used for compression and blood flow moderation in a variety of applications including, but not limited to, blood flow restriction training (BFR) and limb compression for injury prevention.

### BACKGROUND OF THE INVENTION

The effect of occlusion training or blood flow restriction (BFR) as occlusion training is alternatively known, is to cause a variety of metabolic processes including the buildup of lactic acid in the muscle which contribute to muscle growth without the need for the user to lift normal loads typically required to stimulate a muscle to grow. In this example of a metabolic process facilitated by BFR training the band and air assembly set to the correct pressure prevent lactic acid from being washed away by blood and out of the target limb. The buildup of lactic acid in the muscle is picked up by receptors in the muscle and has been shown cause the brain to secrete growth hormone which aid hypertrophy and protein synthesis. A state is created within the muscle that stimulates a growth response that would normally be experienced after very high intensity training.

### SUMMARY OF THE INVENTION

This invention is directed to apparatus for compression and blood flow moderation, and methods of using such apparatus. In accordance with one embodiment, the apparatus comprises a length-adjustable, flexible band adapted to encircle a human extremity. The band has an outer surface and an inner surface, and the inner surface includes an elongated inflatable bladder. A manually operated pump communicates with the inflatable bladder through a one-way valve, causing the bladder to inflate with air and constrict blood though through the extremity. A manually operated pressure-relief mechanism is provided to deflate the inflatable bladder, either to remove the band or ‘fine-tune’ the amount of pressure applied by the band through the inflatable bladder.

In one embodiment, the manually operated pump is integral to the band and accessible on an outer surface thereof. The manually operated pump may be a compressible bulb operated with a finger, for example. Alternatively, the manually operated pump may be disposed remotely from the band, with the pump being in pneumatic communication with the bladder through a length of flexible tubing. The pressure release mechanism may also be integral to the band or remotely located. The one-way valve may be a duckbill or a ‘cuspid’ valve.

In certain embodiments the band may be temporarily or permanently affixed to a garment such as a shirt, shorts or pants. In each instance, the manually operated pump may be disposed remotely from the band on the garment, with flexible tubing being used to interconnect the manually operated pump to the band. As examples, the band may be temporarily or permanently affixed to the sleeve of a shirt and adapted to encircle an upper arm forearm, in which case the manually operated pump may be disposed near the lower edge of the shirt. Alternatively, the band may be temporarily or permanently affixed to the shorts or pants and adapted to encircle an upper thigh, in which case the manually operated pump may be disposed proximate to a waistband.

Preferred embodiments may incorporate two bands, each temporarily or permanently affixed to a sleeve of a shirt and each being adapted to encircle a different upper arm forearm or temporarily or permanently affixed to shorts or pants, each band being adapted to encircle a different upper thigh. Such embodiments may include two manually operated pumps, either on respective bands, or disposed remotely from, and interconnected to, a respective one of the two bands through separate lengths of flexible tubing.

An alternative embodiment may include a pressure sensor in communication with a pressure display operative to display the pressure in the inflatable bladder. The pressure display may be associated with the band—on the band or pressure relief valve, for example, or disposed remotely from the pressure sensor, with electronics being operative to transmit a signal indicative of bladder pressure to a remote display. Such a signal may be a wireless signal received by a smartphone including the remote display.

### BRIEF DESCRIPTION OF THE DRAWINGS

FIG. 1A is an outer surface view of an occlusion band according to the invention;

FIG. 1B is a side view of the occlusion band of FIG. 1A;

FIG. 1C is an underside view of the occlusion band of FIG. 1A;

FIG. 1D show the occlusion band of FIG. 1A in a fastened condition as would be the case when the band encircles a body part such as a limb;

FIG. 1E is a different view of the occlusion band of FIG. 1A in a fastened condition;

FIG. 2 is a side view of an alternative embodiment of the invention illustrating different optional features such as an air relief valve that may include a mechanical or electronic gauge which displays the current pressure of the inflatable bladder on an extremity;

FIG. 3A is an oblique diagram of an air assembly and subcomponents which allow for the moderation of air flow into a band, compression sleeve, or an article of clothing;

FIG. 3B shows the system of FIG. 3A in cross section;

FIG. 4A is a diagram that show an air assembly similar that of FIGS. 3A, B, but including a relief valve similar that that used in conventional blood pressure cuffs.

FIG. 4B is an alternative view of the assembly of FIG. 4A;

FIG. 5 illustrates the way in which one or more air assemblies with separate pumps and relief valves may be sewn into the clothing in an area providing for easier access and adjustment, for example, in the lower seam of a shirt;

FIG. 6 illustrates an embodiment using components similar to those of FIG. 5, but integrated into a pair of shorts;

FIG. 7 shows an embodiment of the invention integrated into a pair of tights with pump(s), air line 704, and occlusion band(s); and

FIG. 8 depicts a compression sleeve embodiment including an integrated air assembly and an inwardly facing bladder.

### DETAILED DESCRIPTION OF THE PREFERRED EMBODIMENTS

This invention is directed to flexible air bladders and associated air assemblies used for compression and blood flow moderation in a variety of applications including, but not limited to, blood flow restriction training (BFR) and limb compression for injury prevention. The apparatus includes a flexible synthetic air bladder and an integrated air assembly for manually adding and removing air from the bladder to moderate pressure of the bladder in conjunction with a body part on the user's skin. With the synthetic band and integrated pump mechanism disclosed herein, a user may achieve growth response without the strain of heavy loads and the associated longer recovery times.

Preferred embodiments include an integrated air assembly comprising a silicone or urethane pump, a check valve air intake, a duckbill valve, and a pressure release valve. The user can properly position and fasten the air bladder around a limb using a special type of band, sleeve, or by wearing a piece of clothing containing one or more integrated, strategically placed air bladders and subsequent integrated air assemblies.

The air assembly allows the user to easily and manually add air to the bladder by pressing on a designated area of the pump which causes the bladder to extend on the inward facing portion of the band, compression sleeve, or piece of clothing toward the limb until a desired pressure is achieved around the limb to support blood flow moderation. The integrated air assembly may contain a pressure control component which allows the user to directly and manually use a release valve to remove air from the bladder in order to adjust the system to apply an appropriate amount of pressure to the limb for the user's desired outcome.

Once the air bladder and associated air assembly have been secured and the desired pressure applied, the user may begin exercise where the user benefits from the effects of partial occlusion and blood flow restriction provided by the band and the air bladder pressing against the skin at the user's preferred pressure as provided herein. The system may also be used to apply and adjust compression to any specific area of a limb such as a knee or elbow.

By applying an appropriate amount of pressure in the correct body locations using the bladder and the integrated air pump, the user may limit blood flow from the veins leading out of the muscle groups in the target limb. The band and air bladder in conjunction with each other allow the user to safely place appropriate pressure against veins and effectively restricts or occludes venous return of blood, a process that may build muscle mass and strength while utilizing less weight than otherwise necessary for the same stimulus. The slightly flexible nature of the band allows the muscle group to expand and contract without losing the necessary tension to restrict the return of venous blood flow to the heart.

With the appropriate pressure as gauged by the pump, the pressure release valve, and the user's perception the invention allows the user to be precise in setting the pressure around a limb in a way that the occlusion does not restrict arterial flow, only venous return flow increasing the users control over the perceived tightness of the bladder on the limb. This increased control helps the user engage in the method of practical BFR training, which requires the user to occlude venous return of blood to the heart without affecting arterial flow into the limb. Without the devices and methods described in this disclosure, the user would not have such direct control over the pressure to make small adjustments to the perceived tightness needed to achieve flowthe above method.

The integrated nature of the system also gives the user more control over where and how pressure is applied to the limb, allowing for placement in various types of compression sleeves. This increased versatility allows the user to moderate blood flow with a sleeve affixed to a localized area of a limb such as a joint. Rather than the pressure being applied by the weave of a synthetic or natural flexible fabric, the user benefits from the increased comfort afforded by addition of an inward facing air bladder in which the user can moderate the pressure and therefore the blood flow underneath the sleeve.

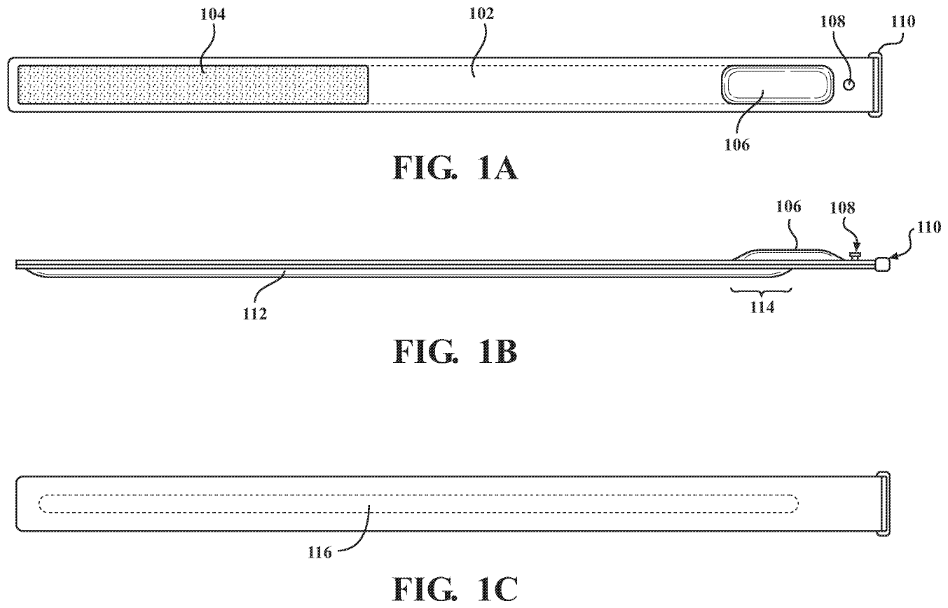

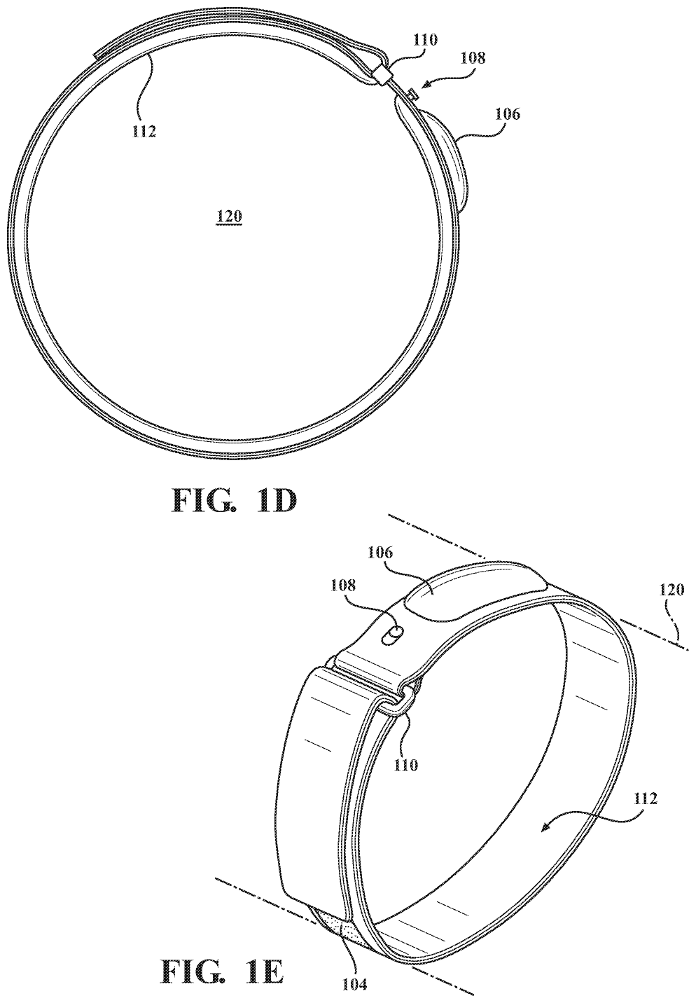

Now making reference to the accompanying drawings, FIGS. 1A-E depict an occlusion band embodiment of the invention, with FIG. 1A being an outer surface view; FIG. 1B is a side view; and FIG. 1C is an underside view, all with the band in a flattened or outstretched state. FIGS. 1D, E show the band in a fastened condition as would be the case when the band encircles a body part such as a limb. Accordingly, the length of the band would be dimensioned so as to achieve a circumference in the range of 10 to 36 inches, to suit the upper forearm of a thinner user to the upper thigh of a body builder, for example. Bands of different lengths and widths might preferably be provided for different applications and placements.

The occlusion band 102 and other components may be constructed from natural or synthetic materials, such as leather, silicone or other polymeric materials, with or without fibrous reinforcement(s). As seen in FIG. 1A, the outer surface includes a strip of hook-and-loop material 104 and a flexible, manually operated bulb-type pump 106, both stitched to the band 102. The drawing also shows air-pressure release valve 108, and D-ring 110 to receive the opposing end of the band with hook-and-loop material that folds back on itself. Note that while hook-and-loop fastening is preferred, other connection mechanisms such as hooks with spaced-apart eyelets may alternatively be used. Note that in this and all embodiments disclosed herein, the pump and pressure release valve may be separate, as shown, or integrated into a single unit.

The side view of FIG. 1B shows cuff air channel 112 which is in pneumatic communication with bladder 106 through overlapping region 114. More specifically, the bladder 106 communicates with elongated bladder air channel, evident of underside view of FIG. 1C. The interface between air pump 106 and bladder air channel forms a one-way check valve, such that as air is pumped into bladder 112 is one direction only, enabling pressure to gradually build with each compression of pump 106. Such a one-way valve may be implemented in different ways, including a duckbill valve, bicuspid or tricuspid type valves, and so forth.

In use, the band 102 is wrapped around a user's extremity 120, and secured back on itself using a hook-and-loop fastener as shown in FIGS. 1D, E, to achieve an initial tightness. Such extremity may for example be the upper arm, the upper thigh, the upper calf, or the upper forearm. In practice, two bands 102 may be attached to the body, one band for each limb. The band 102 exhibits adequate elasticity to move as the muscles of interest move during exertion while remaining adequately tight in order to restrict venous return without restricting arterial flow.

The integrated nature of the air assembly in the band is used to moderate blood flow during exercise. Once the wrapping of the band about the extremity of interest is complete, the user may then press the pump 106 in order to fill the bladder 112, which presses inwardly on the user's skin until the desired pressure is reached. The air relief valve 108, a subcomponent of the air assembly, may be used to make fine adjustments to the pressure in the bladder 112 until the user subjectively feels the appropriate pressure on the extremity.

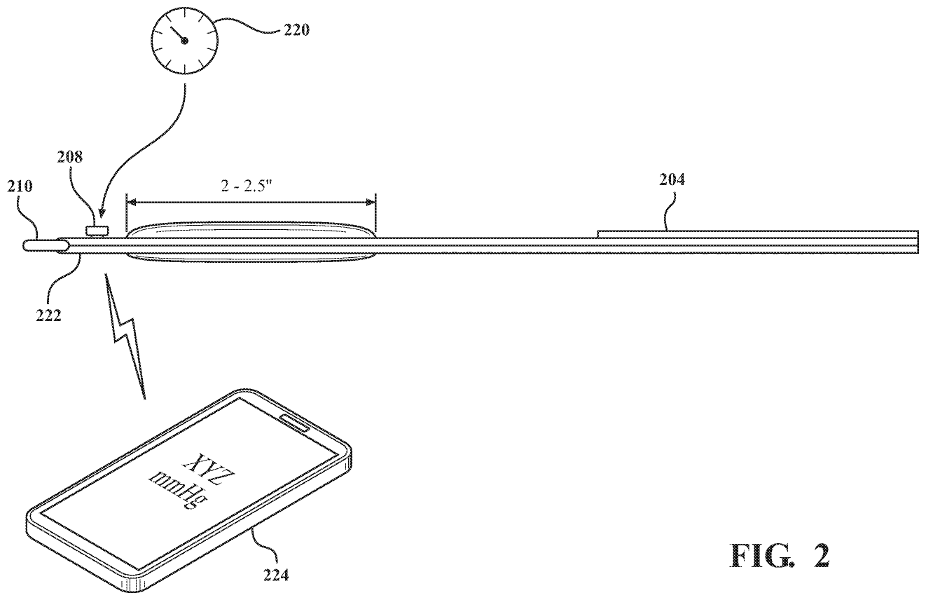

FIG. 2 is a side view of an alternative embodiment illustrating different optional features. For one, the outer surface of the air relief valve 208 may include a mechanical or electronic gauge 220 which displays the current pressure of the bladder on the extremity in millimeters of mercury (mmHg). A separate gauge (not shown) may also be provided, separate from the air relief valve 208. As a further alternative, the current pressure in mmHg may be transmitted by integrated electronics 222 to a smartphone application 224. Such electronic would include an electronic pressure sensor, microcontroller and Bluetooth interface, all battery powered. A portion of hook-and-loop strip is depicted at 204, and D-ring is shown at 210.

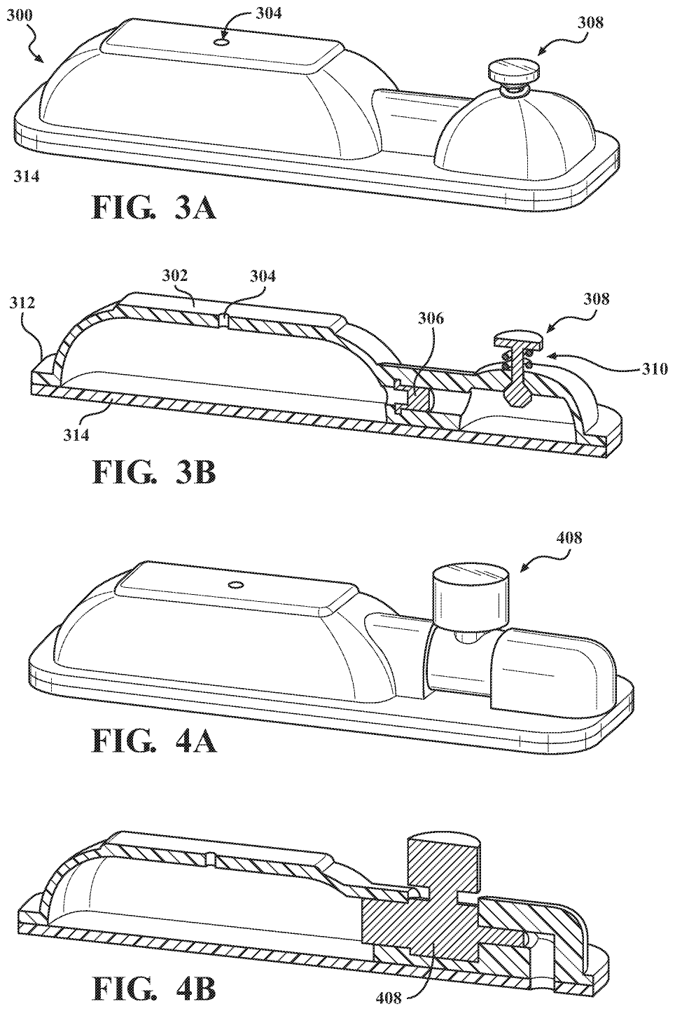

FIGS. 3A, B are oblique diagrams of an air assembly and subcomponents which allow for the moderation of air flow into a band, compression sleeve, or an article of clothing. FIG. 3B shows the system in cross section. This embodiment is comprised of a molded body 300, a check valve air intake 304, a one-way valve 306, and a relief valve 308. In preferred embodiments, valve 306 is a duckbill valve inserted after molding body 302, and valve 308 is a molded or machined poppet valve with spring 310.

The molded body 300 includes an upper body 302 rests on a stamped lower body 112, both made of material such as urethane or silicone having a sufficiently high durometer to maintain structure while still being pliable enough to be comfortably integrated and worn in a band, compression sleeve, or piece of clothing. The upper body 302 allows a user to place their finger over the check valve intake 304 and press down, pushing the air through the one way duckbill valve 306, and through the passage outlet to bladder 314, so as to moderate pressure on the extremity blood flow into and out of an extremity.

FIGS. 4A, B are diagrams that show an air assembly similar that of FIGS. 3A, B, but including a relief valve 408 similar that that used in conventional blood pressure cuffs. In this embodiment, the relief valve integrates a cuff dial that contains the duckbill valve. This allows the user has an additional option to release air from the bladder in a slow, controlled manner to make more precise adjustments and reset the pressure during use.

The various air assemblies according to the invention may be integrated into an article of clothing for the purpose of blood flow restriction training. For example, the band of FIGS. 1A-D may be sewn into the upper arm or upper thigh regions surrounding the target extremity so that it may be inflated and deflated in a shirt or pants without requiring the user to physically apply, transport, or store an external set of bands.

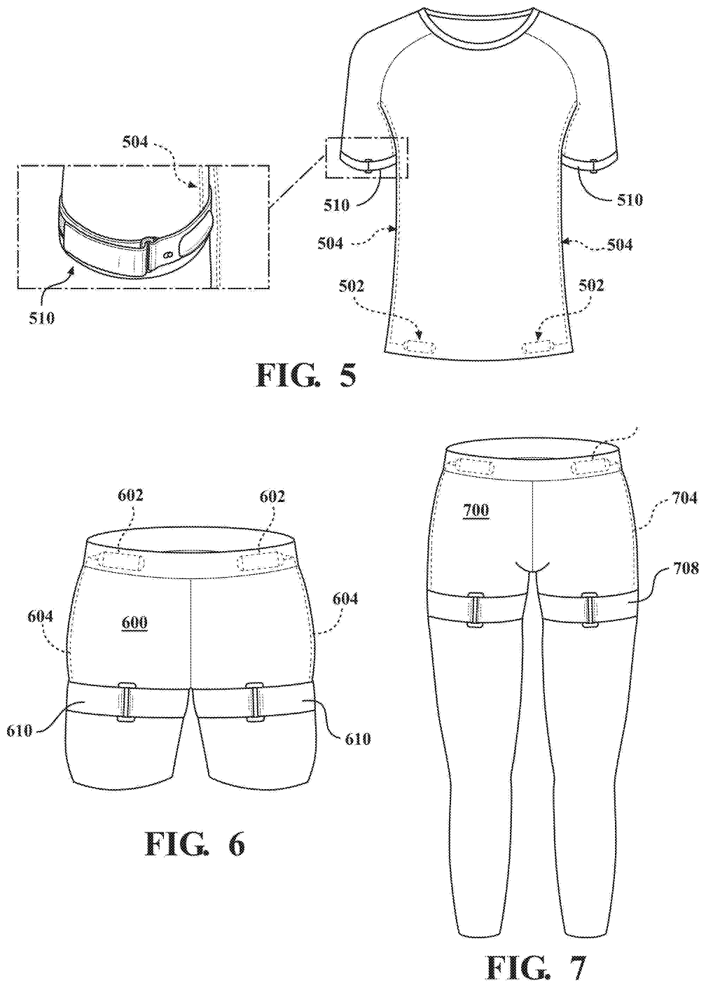

Alternatively, as shown in FIG. 5, one or more air assemblies 502 (with separate pumps and relief valves) may be sewn into the clothing in an area providing for easier access and adjustment, for example, in the lower seam of a shirt, as shown. Air line(s) 504 connects each respective pump/relief valve assembly 502 to bladder(s) 510 in the sleeve placed adjustable band 101.

FIG. 6 illustrates a system using components similar to those of FIG. 5, but integrated into a pair of shorts 600. Pump and relief-valves assemblies 602 may be placed near the waist, with pneumatic lines 604 feeding separate bands 610 positioned in the upper-thigh regions of the wearer. As with the embodiment of FIG. 5, each band would incorporate its own hook-and-loop (or other fastening mechanism) to set an initial tightness prior to pump activation. FIG. 7 shows the invention integrated into a pair of tights 700, with pump(s) 702, air line(s) 704, and band(s) 708.

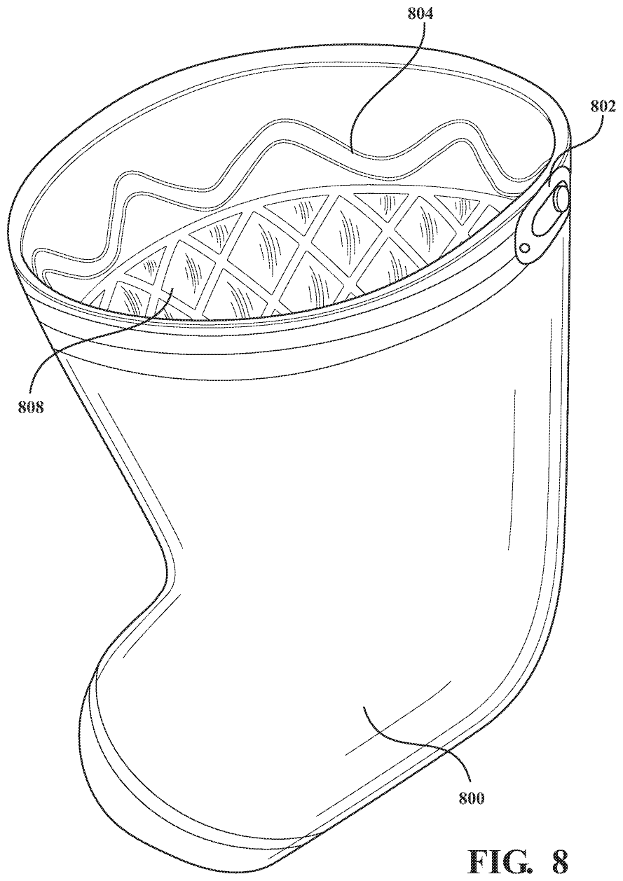

The embodiment of FIG. 8 comprises a compression sleeve 800, an integrated air assembly 802, and an inwardly facing bladder 808. In this embodiment, the sleeve 800 would be strategically placed on a portion of a limb, such as a joint requiring pressure and support, rather than at the upper portion of the extremity. The bladder 808 is fashioned in a way that consistent pressure may be inwardly applied and adjusted by the air assembly 802 around the joint in a uniform way to moderate the flow of blood in the localized area. Consistent with the rest of this disclosure, the air assembly 802 may then be used to make minute, manual adjustments to the pressure of the sleeve 800 and bladder 808 around the joint. The inside of the sleeve 800 may include one or more no-slip textured surfaces 804 to keep the sleeve and bladder in a desired position on the joint.

1. Apparatus for compression and blood flow moderation, comprising:
a length-adjustable, flexible band adapted to encircle a human extremity;
wherein the band has an outer surface and an inner surface, and wherein the inner surface includes an elongated inflatable bladder;
a manually operated pump in communication with the inflatable bladder through a one-way valve, whereby operation of the pump causes the bladder to inflate so as to constrict blood flow through the extremity; and
a manually operated pressure-relief mechanism operative to deflate the inflatable bladder.

2. The apparatus of claim 1, wherein the manually operated pump is integral to the band and disposed on the outer surface thereof.

3. The apparatus of claim 1, wherein the manually operated pump is a compressible bulb.

4. The apparatus of claim 1, wherein:
the manually operated pump is disposed remotely from the band; and
the pump is in pneumatic communication with the bladder through a length of flexible tubing.

5. The apparatus of claim 1, further including a pressure release mechanism is integral to the band.

6. The apparatus of claim 1, wherein the band is temporarily or permanently affixed to a garment.

7. The apparatus of claim 6, wherein:
the manually operated pump is disposed remotely from the band on the garment; and
flexible tubing interconnecting the manually operated pump to the band.

8. The apparatus of claim 6, wherein the band is temporarily or permanently affixed to the sleeve of a shirt and adapted to encircle an upper arm.

9. The apparatus of claim 7, wherein:
the band is temporarily or permanently affixed to the sleeve of a shirt and adapted to encircle an upper arm;
the shirt has a lower edge; and
the manually operated pump is disposed near the lower edge.

10. The apparatus of claim 6, including two bands, each temporarily or permanently affixed to a sleeve of a shirt and each being adapted to encircle a different upper forearm.

11. The apparatus of claim 10, including two manually operated pumps, each disposed remotely from, and interconnected to, a respective one of the two bands through flexible tubing.

12. The apparatus of claim 6, wherein the band is temporarily or permanently affixed to the shorts or pants and adapted to encircle an upper thigh.

13. The apparatus of claim 7, wherein:
the band is temporarily or permanently affixed to shorts or pants and adapted to encircle a thigh;
the shorts or pants have a waistband; and
the manually operated pump is disposed proximate to the waistband.

14. The apparatus of claim 6, including two bands, each temporarily or permanently affixed to shorts or pants, each band being adapted to encircle a different upper thigh.

15. The apparatus of claim 14, including two manually operated pumps, each disposed remotely from, and interconnected to, a respective one of the two bands through flexible tubing.

16. The apparatus of claim 1, wherein the one-way valve is a duckbill or cuspid valve.

17. The apparatus of claim 1, further including a pressure sensor in communication with a pressure display operative to display the pressure in the inflatable bladder.

18. The apparatus of claim 1, wherein:
the pressure display is disposed remotely from the pressure sensor; and
electronics operative to transmit a signal indicative of bladder pressure to the remote display.

19. The apparatus of claim 18, wherein the signal is a wireless signal.

20. The apparatus of claim 19, wherein the signal is a wireless signal is received by a smartphone including the remote display.

21. The apparatus of claim 1, including a compression sleeve into which the band is integrated.
